(19) United States
(12) Patent Application Publication
(10) Pub. No.: US 2018/0182473 A1
(43) Pub. Date: Jun. 28, 2018

(54) RESPIRATION SYSTEM AND METHOD

(71) Applicant: Loewenstein Medical Technology S.A., Luxembourg (LU)

(72) Inventor: Matthias SCHWAIBOLD, Karlsruhe (DE)

(21) Appl. No.: 15/854,029

(22) Filed: Dec. 26, 2017

(30) Foreign Application Priority Data
Dec. 23, 2016 (DE) 102016015440.3

(51) Int. Cl. G16H 10/60; A61B 5/08; G16H 20/30; G16H 15/00

(52) U.S. Cl. CPC G16H 10/60; A61B 5/0816; A61B 5/087; G16H 15/00; G16H 20/30

(57) ABSTRACT

Disclosed is a method for operating a data processing device of a respiration system comprising a respirator machine and a remote station physically separated from the respirator machine. Therapy data is transmissible by the data processing device between the respirator machine and the remote station and at least one authorization code is stored by a user of the respiration system in the data processing device to obtain an authorized access to the therapy data via the remote station. In this process, the authorization code is only provided to the user when the latter requests the authorization code by a user action to be performed vis-à-vis the data processing device.

### CROSS-REFERENCE TO RELATED APPLICATIONS

The present application claims priority under 35 U.S.C. § 119 of German Patent Application No. 102016015440.3, filed Dec. 23, 2016, the entire disclosure of which is expressly incorporated by reference herein.

### BACKGROUND OF THE INVENTION

### 1. Field of the Invention

The present invention relates to a method for operating at least one data processing device of a respiration system as well as a respiration system with at least one data processing device. The respiration system comprises at least one respirator machine and at least one remote station physically separated from the respirator machine. With the data processing device, therapy data are transmitted between the respirator machine and the remote station.

### 2. Discussion of Background Information

Respirator machines or sleep therapy machines generally have a storage device, in order to save and call up therapy data. Thus, e.g., the respiration parameters are stored which are needed to treat a particular respiratory disorder. Often data are also stored which characterizes the course of the therapy.

Such data are especially helpful in judging the success of the treatment or supporting a particular diagnosis. Furthermore, the data also provide information as to whether the user is properly and regularly using the respirator machine. In order to assure a reliable diagnosis and an effective therapy, a regular and as frequent as possible evaluation of the therapy data is therefore especially important.

Therefore, respirator machines have become known whose memory can be accessed from the outside, so that no visits to the doctor are needed in order to appraise the therapy data. This is especially advantageous for respiration systems with several respirator machines or patients.

For example, an access to the stored data is possible as long as a remote station with suitable software is available, which can interpret the data. However, this constitutes a serious weakness in terms of data protection and data security.

Therefore, for certain respirator machines it is also necessary to enter the serial number of the machine, in order to gain access from the remote station. This is supposed to ensure that the machine with the entered serial number is physically located at the user. However, this does not always sufficiently ensure that there is no mistake in the entry or that the user would like to gain access to a machine for which he has no authorization.

Therefore, in the case of certain respirator machines oftentimes a second number is printed on the machine as a machine code. A machine access can only be set up if the serial number and the machine code are properly entered together. However, this solution also has substantial drawbacks. Persons who had the machine in front of them at an earlier time could note down or photograph the machine code. Persons who have the machine in front of them at a later time can also gain access unnoticed, whether or not they are authorized.

In view of the foregoing, it would be advantageous to have available a method for operating a data processing device of a respiration system and such a respiration system with which an improved access to therapy data is possible. In particular, the access should be improved in terms of data protection and data security.

### SUMMARY OF THE INVENTION

The present invention provides a method for operating at least one data processing device of a respiration system comprising at least one respirator machine and at least one remote station physically separated from the respirator machine. Therapy data are at least partly transmissible by the data processing device between the respirator machine and the remote station and at least one authorization code is stored by at least one user of the respiration system in the data processing device to obtain authorized access to the therapy data via the remote station. The authorization code is only provided to the at least one user when the latter requests the authorization code by at least one user action to be performed vis-à-vis the data processing device.

In one aspect of the method, the user action may be performed at least in part with the respirator machine, and the authorization code only authorizes an access to the therapy data of that respirator machine with which the user action is performed.

In another aspect, the user action may comprise at least one placement of the respirator machine in operation and preferably, also at least one entry in the respirator machine.

In yet another aspect, the user action may comprise at least one biometrical detection of the user and/or at least one voice command and/or at least one connecting of the respirator machine to at least one storage medium.

In a still further aspect, the requested authorization code may be indicated to the user on at least one display device and/or may be stored on at least one storage medium.

In another aspect, the user action may comprise at least one registration of the user vis-à-vis the data processing device, preferably vis-à-vis the remote station, so that only a registered user can request the authorization code.

In another aspect, the user upon registration may be assigned at least one certificate and the certificate must be presented to the respirator machine in order to be able to request the authorization code. The certificate may be stored on a portable storage medium, and the authorization code may only be requested when the respirator machine is connected to the storage medium. Further, the remote station in addition to the certificate may also store at least one device certificate identifying the respirator machine on the storage medium.

In another aspect of the method, the authorization code may be generated at least partly by the respirator machine with the help of the certificate.

In another aspect, access to therapy data may be released when a registered user logs into the remote station after having first requested the authorization code as a registered user.

In another aspect, access to therapy data may only be released when the at least one user has communicated the authorization code provided to her to the remote station.

In another aspect of the method, the authorization code may be entered by the at least one user in the remote station.

In another aspect, the respirator machine may relay the authorization code at least partly independently to the remote station.

In another aspect, the authorization code may comprise at least one serial number and/or at least one device identification number of the respirator machine or may be generated at least in part from the latter.

In another aspect, the authorization code may comprise at least one piece of information about an authorization of the user with respect to access to therapy data and/or at least one piece of information about at least one read right and/or a write right of the user.

In another aspect, the authorization code may have at least one time-limited validity and/or a onetime validity.

In another aspect, the validity of the authorization code may be associated with the user.

In another aspect, the validity of the authorization code may be abolished when the respirator machine is used for a new patient.

In another aspect, the authorization code which is requested by the at least one user may be at least partly stored in the respirator machine and/or may be at least partly generated by the respirator machine.

In another aspect of the method, at least one portion of the authorization code which is requested by the user may be physically separated from the respirator machine, and may, in particular, be generated and/or stored in the remote station.

The present invention also provides a respiration system which comprises at least one respirator machine comprising at least one respirator device to create an air flow for a respiratory therapy, at least one remote station physically separated from the respirator machine, and at least one data processing device capable of sending therapy data between the respirator machine and the remote station. At least one authorization code can be stored in the data processing device in order to grant a user authorized access to the therapy data via the remote station, and the data processing device is suitable and designed for providing the user the authorization code when the latter requests the authorization code by at least one user action to be performed vis-à-vis the data processing device.

The method according to the invention serves for the operating of at least one data processing device of at least one respiration system. The respiration system comprises at least one respirator machine and at least one remote station physically separated from the respirator machine. By the data processing device, therapy data are at least partly transmissible between the respirator machine and the remote station. By at least one user of the respiration system, at least one authorization code is stored in the data processing device in order to obtain an authorized access to the therapy data via the remote station. The authorization code is only provided to the user when the latter requests the authorization code by at least one user action to be performed vis-à-vis the data processing device and especially vis-à-vis the respirator device and/or the remote station.

In particular, the action by the remote station requires that the user has at least one authorized user account with the remote station and in particular has previously authenticated herself there. The authorization code provided preferably identifies the user uniquely.

The method according to the invention offers many advantages. One major advantage is that the authorization code is only provided to the user when the latter requests it and performs at least one user action for this. This makes possible a much more reliable access to the therapy data in terms of data protection and data security. Thus, the authorization code is not visible from the outside on the machine before the machine is placed in operation and the output of the authorization code is requested.

In particular, the user obtains with the aid of the authorization code an access to the respirator machine and preferably to the therapy data stored there. In particular, the user may view and/or process therapy data stored on the respirator machine from the remote station, for example, evaluate, change, copy and/or move the data.

Without authorization, in particular, no access to the therapy data is possible. In particular, an access to the therapy data is only possible by using the authorization code made available.

If the user has an authorized access, then for example a direct access can be provided to the therapy data which are stored on the respirator machine. But the therapy data may also be stored at least temporarily in the remote station and for example on a server, so that the access to the therapy data of the respirator machine occurs indirectly via the remote station.

The storing of the authorization code in the data processing device may occur at least in part manually by the user, e.g., by entry on at least one user interface and/or by connection and/or inserting of a data medium on the respirator machine which contains the authorization code. It is also possible for the storage of the authorization code to occur at least partly automatically, for example by at least one network transmittal of the authorization code to the respirator machine and/or the remote station and/or by at least one generating by at least one algorithm in the respirator machine. In particular, the data processing device is operatively connected to the respirator machine and the remote station, so that the storing of the authorization code in the data processing device can occur by storing the authorization code in the respirator machine and/or the remote station.

The authorization code in particular cannot be viewed by the user without the request undertaken by the user action. In particular, the authorization code cannot be seen from outside the respirator machine and in particular it cannot be printed on the respirator machine.

In particular, the data processing device is operatively connected to the respirator machine and the remote station, so that a user action performed vis-à-vis the respirator machine or the remote station is also performed vis-à-vis the data processing device.

Especially preferably, the user action is performed at least in part with the respirator machine. In particular, the authorization code only authorizes an access to the therapy data of that respirator machine with which the user action is performed. In this way, for example, it may be ensured that the user also can only access the respirator machine of the respiration system with which he also actually has dealings with. In particular, for this, the authorization code is assigned at least partly to the respirator machine. The assignment is done for example by a serial number and/or a machine identification number or the like. In addition, the authorization code may be assigned to the user.

In particular, the authorization code only authorizes an access to a particular respirator machine and/or only to a single respirator machine. But it is also possible for the authorization code to also authorize an access to other respirator machines of the respiration system. In particular, for this, certain respirator machines are assigned to a group, wherein the authorization code authorizes an access to the respirator machines of the group. This is of great benefit for example for operators of large respiration systems.

The user action involves in particular at least one placement of the respirator machine in operation. Preferably, the user action involves at least one entry in the respirator machine. For the entry, the respirator machine preferably has at least one user interface. The entry may involve, for example, the selection of a machine function which starts the output or generating of the authorization code. For example, a menu entry and/or a text entry may be provided. It is also possible for the user action to involve an entry at least in part via-à-vis the remote station and in particular at least one entry in a user interface of the remote station.

The user action may involve at least one biometrical detection of the user and/or at least one voice command and/or at least one connecting of the respirator machine to at least one storage medium. The biometrical detection may involve, for example, at least one detection of at least one fingerprint and/or at least one iris structure. In particular, at least one portable or removable storage medium is connected to the respirator machine. Also at least one computer or smartphone, tablet computer or the like may be connected by cable or wirelessly, e.g., WiFi, Bluetooth etc., to the respirator machine. In particular, the storage medium contains at least one specific data set, such as a certificate, by which the authorization code is generated and/or retrieved.

In one advantageous embodiment, the requested authorization code is indicated to the user on at least one display device, especially the respirator machine. The display device comprises for example a display or is designed as such. Thus, it can be assured with reliability that the respirator machine is also in front of the intended user when the access is arranged.

It is also possible for the requested authorization code to be stored on at least one storage medium, especially a removable one. In this way, the user may store the authorization code on the remote station in an especially comfortable manner and avoid data entry mistakes.

Preferably, the user action involves at least one registration of the user vis-à-vis the data processing device and especially preferably vis-à-vis the remote station. In this way, in particular, the authorization code may only be requested by a registered user. Thanks to such a registration, a particularly high degree of data security or data protection is achieved.

In particular, at least one authentication of the user vis-à-vis the data processing device occurs in the course of the registration. It is also possible for an authorization of the user to occur in the course of the registration. In particular, during the authorization the access rights to particular therapy data are established for an individual user. The data processing device may preferably administer at least two authorization codes of two different users, who may possess different access rights.

For example, at least one user account is set up during the registration in the remote station. In particular, it is also established what kind of access rights the user will obtain. For example, a user account for a caregiver or provider may provide a broader access to therapy data than a user account for a patient.

In particular, the user action involves, besides the registration, also at least one further step in order to be able to request the authorization code. Yet it is also possible for the performance of the registration to already constitute the user action, so that the authorization code is also requested at the same time as the registration.

Upon registration, the user is preferably assigned at least one certificate. In particular, the certificate must be presented to the respirator machine in order to be able to request the authorization code. It is also possible that the certificate must be presented to the remote station in order to be able to request the authorization code. In this way, it may be assured with special reliability that only a particular user is requesting the authorization code. For example, the certificate contains an identification of the user. In particular, the certificate comprises an establishing of the access rights of the user and/or an authorization of the user.

In particular, the certificate has a time-limited validity and/or a onetime validity. It is also possible for the validity of the certificate to be connected to the user. This has the benefit that, for example in event of the loss of a storage medium with a certificate stored in it, the certificate can be declared to be invalid, so that a misuse of the lost storage medium is prevented.

Preferably, the certificate involves an assigning of the user to a user group. In particular, a particular authorization is provided for each user group. For example, doctors or caregivers, providers and patients are assigned to different user groups with different authorizations. Thanks to such a configuration, the respirator machine can recognize with the aid of the certificate how much of an access right should be granted to the respective user.

Especially preferably, the certificate is stored by the remote station on a portable storage medium. In this case, the authorization code may preferably only be requested when the respirator machine is connected to the storage medium and in particular has access to the storage medium.

For example, the certificate is stored on the storage medium when the user registers via the remote station on a server. The user may bring the storage medium with him to the respirator machine and insert it there. With the aid of the certificate, the respirator machine preferably recognizes the user and in particular also his authorization and preferably gives out a suitable authorization code or sends the authorization code or the certificate back to the remote station, which in particular checks the validity and identifies the user.

The certificate may comprise at least one machine certificate identifying the respirator machine and/or at least one user certificate identifying the user, or be designed as such.

Preferably, the remote station stores in addition to the certificate also at least one uniquely identifying machine certificate on the storage medium. In such an embodiment, the certificate is designed in particular as a user certificate. In particular, the machine certificate is read by the respiration system and preferably sent back to the remote station once more. In this way, the remote station can check to make sure it is indeed an authorized respiration system and not a simulation trying to send data to the remote station in order to store falsified, invalid, or invented therapy data there.

Yet it is also possible to provide a non-portable or permanently installed storage medium. Then, for example, a network connection is established between the storage medium with the certificate and the respirator machine in order to be able to request the authorization code.

Especially preferably, the authorization code is generated at least partly by the respirator machine with the help of the certificate. This has the advantage that a user and, for example, his group affiliation or authorization can be identified with the aid of the authorization code. It is also possible for the authorization code to be generated at least partly by the remote station with the help of the certificate.

In particular, the authorization code comprises at least part of the certificate and/or at least part of a machine code of the respirator machine. The machine code contains, for example, at least one serial number and/or at least one machine identification number. In this way, the respirator machine may also be identified particularly well when logging in with the authorization code. A generating of the authorization code independently of the certificate is also possible.

In an especially preferred embodiment, the access to the therapy data is released when the registered user logs in to the remote station after having first requested the authorization code as a registered user. It is thereby especially preferable for the user to have first requested the authorization code by presenting the certificate. Such an embodiment is especially secure and at the same time very comfortable, since the user does not need to enter the authorization code in the remote station.

In particular here, a transmission of the authorization code occurs from the respirator machine to the remote station by at least one network connection or wireless data connection. The transmission in particular is automatic when a registered and preferably certified user requests the authorization code.

In one advantageous embodiment, the access to the therapy data is only released when the user has communicated the authorization code provided to him to the data processing device and especially to the remote station. In this way it can be assured in an especially reliable manner that the person who is trying to access the therapy data from the remote station is also the person who has visited the respirator machine and requested the authorization code there.

Preferably, the data processing device compares the authorization code communicated by the user against a generated and/or stored authorization code. In particular, the access to the therapy data is only released if the comparison shows a suitable match.

In particular, the authorization code is entered by the user in the remote station. In this way, the user communicates the authorization code provided to him to the remote station so that the access to the therapy data can be released. For example, the user notes down the authorization code while he is with the patient with the respirator machine. Afterwards, at his workstation, he establishes a link to the remote station and enters the authorization code there.

But it is also possible to enter the authorization code by at least one voice recognition. The relaying of the authorization code may also involve at least one other manual step, for example, the transporting of the authorization code stored on a storage medium from the respirator machine to the remote station. The entry can also be done in that the user connects the remote station to at least one storage medium on which he has stored the authorization code. An optical entry of the authorization code is also possible, for example as QR code, which is read in particular with the aid of a camera and afterwards evaluated, or the like.

In one advantageous embodiment, the respirator machine relays the authorization code at least partly independently to the remote station. An independent transmission is especially preferred when the respirator machine generates the authorization code at least partly with the aid of a certificate. In this way, it can be assured in a reliable manner that a transmission occurs only to an authorized user.

In particular, the authorization code comprises at least one serial number and/or at least one machine identification number of the respirator machine. It is also possible for the authorization code to be generated at least in part from a serial number and/or machine identification number. The authorization code in particular comprises at least one number and/or at least one letter and/or special character. Preferably the authorization code comprises at least one sequence of at least four characters or more. The authorization code may be at least in part randomly generated.

The authorization code may also comprise at least one defined sequence of sounds and/or gestures and/or movements. In particular, the authorization code comprises at least one gesture and/or movement of at least one finger. The authorization code may also comprise at least one fingerprint and/or at least one image of at least one portion of the iris and/or the retina of the user.

In particular the authorization code contains at least one check point. The check point can be determined in particular with the aid of the serial number and/or machine identification number of the respirator machine and/or the other places of the authorization code. This has the benefit that a check for entry errors can be done already from the respirator machine and/or the remote station before the entire authorization code is transmitted to the respirator machine or the remote station.

Preferably the authorization code comprises at least one piece of information about an authorization of the user with respect to access to the therapy data. For example, the authorization code indicates whether the user is authorized for an access to all therapy data or only for partial access. The authorization code may assign the user to at least one user group. This may be done preferably by the integration of a user type in the authorization code, with the aid of which the remote station can administer the access rights.

The authorization code may contain at least one piece of information about at least one read right and/or a write right of the user. Preferably, the read and write rights are assigned to a user in the course of the registration of the user and especially preferably with the aid of the certificate of the user. For example, the certificate contains at least one read right and/or write right.

Preferably the data processing device or the respiration system also distinguishes between read and write rights in the access rights. Read rights are required in particular in order to be able to read out therapy data of the respiration system at the remote station. Write rights are required in particular to be able to change therapy data of the respiration system at the remote station, e.g., the respiration settings, especially at least a respiration pressure or a pressure limit, respiration rate, or a humidifier line.

Especially preferably it is provided that write rights can basically be blocked at the data processing device or at the respiration system itself by means of the data processing device. This is done, e.g., by an appropriate entry at the user interface, a voice command or an entry by cable or storage medium. This means an especially good security. For example, if it is determined that a remote adjustment of the respiration system by authorized users of the remote station should not be possible in any way, this will prevent unauthorized users or an unauthorized simulation of the remote station from carrying out a remote adjustment.

In all configurations it is especially preferable for the authorization code to have at least one time-limited validity and/or a onetime validity. In particular, with the expiration of the validity the authorized access to the therapy data will also end. The authorization code may be valid only for a onetime access. It is also possible for the authorization code to be valid only to establish at least one access. The access so established may then also be valid for the long term, even if the authorization code itself is no longer valid.

It is also possible for the authorization code to have a validity which is limited to the duration of a scheduled usage period of the respiration system. This can prevent especially effectively unauthorized access to respirator machines no longer in use. In particular, when there is a change of patients, then at least one new authorization code must be requested.

Preferably the authorization code is valid only for a particular user and especially only for a registered user. It is also possible for the authorization code to be valid for a number of users and, for example, for a user group.

In particular, the validity of the authorization code is connected to the user. Preferably, the validity of the authorization code is connected to the authorization of the user. The connecting is done in particular by assigning of the authorization code to a registration or a user certificate. For example, it is possible for the validity of an authorization code to end when the authorization of the user is restricted and/or broadened. This ensures that the access rights which are released by the authorization code also correspond to the authorization of the user. In this way, one can prevent confidential therapy data being viewed by users who are only allowed access to general data.

Especially preferably, the validity of the authorization code and especially also that of the access is abolished at least for certain users when the respirator machine is used for a new patient. For example, the data processing device monitors at least one characteristic parameter for an assigning of the respirator machine to a patient. This parameter may be, for example, a patient's name and/or address. Then the authorization code will lose its validity, for example, or has to be renewed when such a parameter is changed. A change in patients can also preferably be recognized indirectly by the remote station and the respiration system, e.g., upon deletion of the therapy data in the respirator machine or after a lengthy pause in the use of the respirator machine, preferably of more than one week.

Preferably the authorization code which is requested by the user is at least partly stored in the respirator machine and/or is at least partly generated by the respirator machine. For this, at least one logic and/or at least one algorithm is stored in the respirator machine. In particular, the remote station is also suitable and designed for generating the same authorization code so that a comparison is possible. For example, the same logic or the same algorithm is stored for this both in the remote station and in the respirator machine. The same authorization code may also be stored in the remote station to make possible a comparison. The authorization code may also be sent via a network connection from the respirator machine to the remote station in order to make possible a comparison.

It is also possible that at least one portion of the authorization code which is requested by the user is physically separated from the respirator machine and preferably generated and/or stored in the remote station. The authorization code may also be generated at least partly at a location physically separated from the respiration system, where at least one code generator is situated. For example, the data processing device and preferably the remote station are connected for this by at least one network connection to a code generator. The authorization code can then be sent to the respirator machine, for example, by a network connection or also by the transporting of a storage medium or also by programming during the manufacture, e.g., via a test station.

The respiration system according to the invention comprises at least one respirator machine with at least one respirator device to create at least one air flow for a respiratory therapy. The respiration system comprises at least one remote station physically separated from the respirator machine and at least one data processing device. The data processing device can be used to send therapy data between the respirator machine and the remote station. At least one authorization code can be stored in the data processing device in order to grant a user an authorized access to the therapy data via the remote station. The data processing device is suitable and designed for providing the user the authorization code when the latter requests the authorization code by at least one user action to be performed vis-à-vis the data processing device.

The respiration system according to the invention also offers a better access to the therapy data, so that a high degree of data security and data protection is achieved.

Especially preferably, the respiration system is suitable and designed for being operated by the method according to the invention. The data processing device in particular comprises at least one user interface. The user interface in particular is suitable and designed for receiving a user entry and convert this into an electronically processed signal. The user interface for example comprises at least one human interface device. For example, at least one text entry and/or voice entry can occur via the user interface. With the user interface, a biometrical user detection can also be carried out.

The data processing device is especially suitable and designed for joining together the respirator machine and the remote station by at least one network connection. The data processing device for this may be integrated at least partly in the respirator machine and/or the remote station or be at least partly provided by these. The data processing device may at least partly encompass the respirator machine and/or the remote station. In particular, the data processing device is suitable and designed for reading from and/or writing to at least one storage device of the respirator machine and/or the remote station.

The remote station may be provided by at least one network machine or at least comprise such a machine. For example, the remote station comprises at least one server and/or at least one computer cloud.

The network connection uses in particular a wire-bound connection and/or a mobile radio link and/or the internet, VPN, WLAN (Wireless Local Area Network) and/or LAN (Local Area Network). Corresponding interfaces are preferably provided for this on the respirator machine and the remote station.

### BRIEF DESCRIPTION OF THE DRAWING

Further benefits and features of the present invention will emerge from the description of the exemplary embodiments, which will be explained with reference to the accompanying FIGURE.

FIG. 1 shows a highly schematic representation of a respiration system according to the invention.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawing making apparent to those of skill in the art how the several forms of the present invention may be embodied in practice.

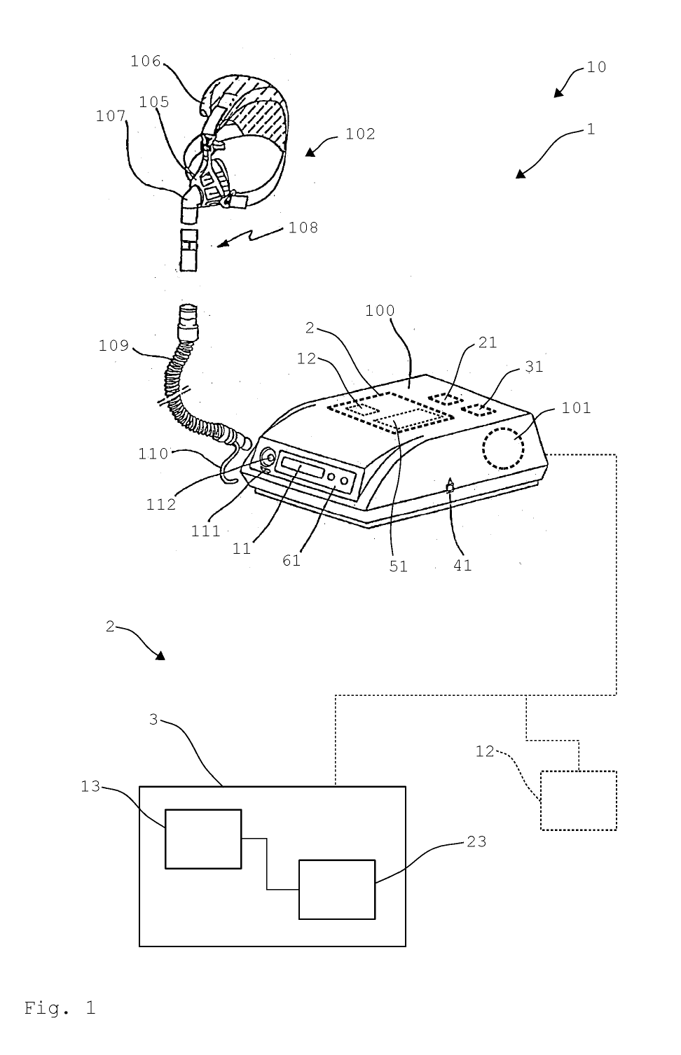

FIG. 1 shows a respiration system 10 according to the invention, which comprises here a respirator machine 1 used as a home respirator machine 11 or a sleep therapy machine. But the respirator machine 1 may also be designed as a clinical respirator machine 1. The respirator machine 1 is suitable and designed for carrying out the method according to the invention.

The respirator machine 1 comprises a respirator device 100 with a blower device 101 to generate an air flow for the respiration. In order to control the respirator device 100 and record therapy data, there is provided here a monitoring device 21. The operation and adjustment of the respirator machine 1 occurs via a user interface 61 with operating elements 103 and a display device 11.

The respirator machine 1 has a breathing interface 102, in order to supply the air flow to a user for the respiration. The breathing interface 102 shown here is a breathing mask 105 configured as a nasal mask. For the attachment of the breathing mask 105 there is provided a headgear 106. The breathing interface 102 may also be configured for example as a full-face mask, a nasal pillow, a tube or a larynx mask.

For the connection of the breathing interface 102 to the respirator device 100 there is provided a connection hose 109, which is joined by means of a connection device 112 to the respirator device 100. By a connection element 107 the connection hose 109 is attached to the breathing interface 102. Between the connection hose 109 and the connection element 107 there is arranged an exhalation element 108, which comprises a valve or is designed as such. The exhalation element 108 is provided in particular to prevent a back breathing into the respirator machine 1 during the user's exhalation.

The monitoring device 21 is operatively connected here to a not otherwise represented sensor device, which comprises one or more sensors for detection of machine parameters and/or patient parameters and/or other characteristic quantities for the respiration.

For example, the monitoring device 21 comprises a pressure sensor, not otherwise shown here, which detects the pressure relations with respect to the breathing interface 102. For this, the pressure sensor is attached by a pressure measuring hose 110 to the breathing interface 102. By an inlet nozzle 111 the pressure measuring hose 110 is attached to the monitoring device 21.

Furthermore, the monitoring device 21 serves here for the actuation of the blower device 101. The monitoring device 21 provides a necessary minimum pressure and compensates for pressure fluctuations due to the breathing activity of the user. For example, the monitoring device 21 also detects the pressure present in the breathing mask 105 and regulates the power of the blower device 101 accordingly, until a desired respiration pressure is present.

The machine parameters needed for the setting of the respirator device 100 and the blower device 101 as well as the machine configuration and/or machine software are stored in a storage device 31.

The monitoring device 21 here may also be designed to detect patient parameters. For this, the monitoring device 21 may be outfitted with sensors for measuring the breathing excursion, for measuring an oxygen saturation of the blood, and/or for measuring an EEG, an EMG, an EOG or an EKG activity.

The respirator machine 1 shown here may be designed as a Fix-Level machine or also as an Automatic-Level machine. In particular, the monitoring device 21 accomplishes a regulation to target machine parameters, which are individually calculated and established in advance with the aid of the characteristic breathing of the user.

It is also possible to adapt the respirator device 100 dynamically and in particular according to the breathing phase of the user. For example, the monitoring device 21 may be used to identify a breathing phase change, so that a higher or lower pressure can be provided according to the breathing phase. For example, the respirator machine 1 may be designed as a CPAP or APAP machine. The respirator machine 1 may also be designed as a Bilevel machine. For example, the respirator machine 1 responds to certain breathing events, such as snoring, shallow breathing, and/or obstructive pressure peaks with corresponding settings of the machine parameters.

The pressure relations detected by the monitoring device 21 are stored together with other machine parameters in a storage device 31. Furthermore, the pressure relations adjusted by the monitoring device 21 or the pressure adaptations undertaken may also be stored as machine parameters in the storage device 31. The detected patient parameters may also be stored in the storage device 31. Furthermore, data on compliance and mask tightness can be stored.

As the machine parameters, it is possible to store for example a starting therapy pressure, a maximum therapy pressure, a minimum therapy pressure and/or a target volume and/or other machine parameters suitable for the setting of the respirator device 100. These machine parameters are retrieved from the storage device 31 by the monitoring device 21 for the setting of the respirator device 100.

Furthermore, the pressure relations detected in the course of the therapy period and/or other machine parameters and/or patient parameters in the course of therapy progress are stored in the storage device 31. As the therapy progress curves, one may register e.g. a flow curve, a pressure curve, and/or an event curve. The therapy progress curves are provided to the storage device 31 by the monitoring device 21, which detects these data during the therapy.

The machine parameters and/or patient parameters and/or therapy curves stored in the storage device 31 are retrieved and evaluated for one or more therapy statistics. The therapy statistics are stored in the storage device 31. In this process, e.g., a mean pressure and/or the therapy duration and/or a leakage parameter may be determined and stored.

The therapy statistics may also be configured as statistics for a therapy period comprising several therapy sessions. The therapy statistics may also involve an evaluation of the willingness of the user to cooperate with the therapy. The values and quantities stored in the storage device 31 as described above are known as therapy data or data in the context of the present invention.

In order to be able to subject the therapy data to a therapeutic or diagnostic analysis, or monitor the functioning of the respirator machine 1, there is provided here a transmittal of the therapy data to at least one remote station 3 by means of a data processing device 2. The data processing device 2 here provides the components or software so that the remote station 3 can interpret the data of the respirator machine 1 and vice versa.

The respiration system 10 may also comprise two or more remote stations 3, which are connected to one or also several respirator machines 1.

The transmittal occurs by means of a transmission device 51 wirelessly and/or wired. The transmittal may occur by one or more cable interfaces, e.g., USB, serial, LAN, data bus, etc.

The transmittal may also occur by one or more wireless interfaces, e.g., mobile radio, LPWAN, Bluetooth, infrared, Sigfox, Lora etc.

The remote station 3 may comprise at least one server 13 and/or at least one personal computer (PC) 23 or also be designed as a computer cloud. Thus, e.g., from a PC 23 one may access a web server 13, which in turn is connected by a network link to one or more respirator machines 1. The data exchange in this case is monitored or controlled by the data processing device 2.

Furthermore, the therapy data may also be stored at least in part on a portable storage medium 41. The storage medium 41 is designed for example as a memory card or a hard disk or a USB mass storage device. The storage medium 41 may be removed from the machine and read out via a reading machine and, e.g., a computer, tablet computer, smartphone etc.

The therapy data may also be read out via a display 11 located in the machine 1 and/or connected to the machine 1 or a user interface 61.

By these same interfaces the machine 1 may be equipped with new configuration data or new program code or functions can be activated in the machine 1. One or more data memories 31, 41 in the machine 1 are thus preferably written into and/or read from the outside.

From the remote station 3 there may occur an accessing of the therapy data from a remote location, so that a location-independent evaluation is possible. Thus, doctors, providers, patients and others may have access to the data in the respiration system 10. The access for data protection and data security reasons is preferably limited to the actually used machine 1. The patient, e.g., only gains access to his machine 1. Technical staff such as providers and doctors gain access, e.g., to the machine 1 of all their patients.

Each user may themselves set up the access to their machines 1. Or one of the users will set up the access and then pass on the authorizations within the data processing device 2 or the respiration system 10 and preferably the remote station 3 to other users. Thus, it must be entered into the data processing device 2 that at least one user wishes and is allowed to have access to one particular machine 1.

In order to organize the data traffic especially securely in terms of data protection and data security, the access here is only possible by using an authorization code. The authorization code is only provided to the user when the latter requests the authorization code. In this way, the code for example cannot be simply read by unauthorized persons from the outside of the machine. In order to request the authorization code, at least one user action must be performed vis-à-vis the data processing device 2.

The retrieval of the authorization code occurs here, e.g., on the display 11 or user interface 61 of the respirator machine 1 or on an outside attached display or by voice output or on a removable storage medium 41. The authorization code here can only be retrieved on the machine 1 during certain operating states of the machine 1.

The operating state in which the authorization code is retrieved is produced here by a user action. For example, the pressing of at least one key/one knob/one touchscreen button on the machine 1 or a combination of keys or other operating elements; entering of a voice command; entry of a storage medium 41 with a particular code, by means of which the machine 1 produces the operating state; entry of a command to the machine 1 by one of the wired or wireless interfaces; checking of a fingerprint or image of the iris or retina of a user.

In one configuration, the operating state is time limited. Thus, the authorization code cannot be read off or read out from the machine 1 for any desired length of time. A preferred time limit is between 5 seconds and 5 minutes. Other periods of time are also possible. Preferably, the operating state can be ended prematurely by the user.

The machine 1 here comprises a logic for generating or retrieving the authorization code with the aid of a serial number or machine identification code. The same logic for generating or retrieving is possessed by the remote station 3, which can read out and display the machine data and write into the data memory 31 of the machine 1 from the outside. Thus, the remote station 3 can check to make sure that the authorization code is correct.

In an alternative embodiment, the authorization code may be generated by a code generator 12, which in turn communicates the authorization code to both the machine 1 and the remote station 3. Thus, the machine 1 can output the correct authorization code and the remote station 3 can check the authorization code for correctness. The code generator 12 may comprise, e.g., at least one random number generator.

In the embodiment shown here, the code generator 12 is part of the respirator machine 1. But the code generator 12 may also be part of the remote station 3. In another embodiment, the code generator 12 may also be connected by a network link to the respirator machine 1 and the remote station 3, e.g., as represented here in broken lines.

The user may also be informed about the correct authorization code in a separate way. Especially preferably in this case the respirator machine 1 enables the retrieval of medical or technical data on a built-in or connected display unit 11 also only when the correct authorization code was entered into the machine 1.

Especially preferably, the authorization code is transmitted in an encrypted or disguised manner during the data communication between the remote station 3 and the respirator machine 1. A transmittal from the remote station 3 to the respirator machine 1 is done so that the machine 1 can output the correct authorization code if the remote station 3 is supposed to recognize the authorization code before the respirator machine 1 in terms of time. A transmittal from the respirator machine 1 to the remote station 3 is done so that the remote station 3 can verify the correct authorization code if the respirator machine 1 is supposed to recognize the authorization code before the remote station 3 in terms of time.

In one embodiment of the invention, the user then enters the requested and provided authorization code to the remote station 3. This is done, e.g., via operating elements 103 and/or via a voice entry and/or with the aid of a storage medium 41, which is connected to the remote station 3.

Only if a valid authorization code is present does the remote station 3 allow a data communication or the storing or displaying or altering of therapy data. This prevents data of the wrong machine 1 being displayed or altered by accident or deliberately.

For example, if the wrong authorization code is entered repeatedly, the access to the respirator machine 1 may be blocked in the remote station 3 for a period of at least one minute. Furthermore, a message will preferably be sent, e.g., to other users or administrators of the remote station 3 or to the respirator machine 1.

The access may be compared to a database of possible machines, e.g., with an ERP system. It is preferably checked whether a provider has acquired the machine 1.

Preferably the remote station 3 distinguishes between different users and allows the communication or storing or displaying or altering of therapy data only for those users who have correctly entered or read in the authorization code.

Especially preferably, several distinguishable authorization codes are generated, which are issued e.g. in different operating states of the machine 1 or communicated to different users by the code generator 12. Thus, depending on the authorization code, the role of the user can be recognized and the access rights limited to a particular subset of the available therapy data.

For example, a patient code and a distinguishable physician code and/or provider code may be provided. Upon accessing of machine data, e.g., via a web server as the remote station 3, the patient will obtain a subset of the memorized data in a view specially prepared for him with explanations optimized for him. Furthermore, he can only alter certain configuration parameters of the respirator machine 1, such as comfort parameters, via the remote station 3.

With the physician code, a different subset or the total set of memorized therapy data is represented in a view specially prepared for physicians. Furthermore, the physician can also change therapeutically relevant configuration parameters, such as at least one therapy pressure, via the remote station 3.

The authorization code in particular is only valid for a selection of respirator machines 1, preferably only for a single machine 1 with a particular serial number or machine identification code.

In an especially preferred embodiment, the authorization code is variable in time. This means that there are a plurality of authorization codes, or a plurality can be generated for the respirator machine 1.

However, only a few of these authorization codes are valid for the current access to the machine 1 or the therapy data received from it, preferably only a single one. A code previously noted down will then no longer be valid. For example, a new authorization code will be generated by the machine 1 for each change of patient or owner.

The time-variable code is created, e.g., in the remote station 3 and transmitted to the machine 1. In this way, it can be reliably assured that the machine 1 is in front of the future user at the moment of organizing an access or during the requesting of the authorization code.

The following embodiments are especially advantageous in the case of time-variable authorization codes.

For example, an authorization code is valid for a lengthy period, which is ended by an event. After this, a new authorization code is valid. Possible events or parameters for changing the valid code may be: expiration of a defined validity time frame; erasing of machine data; assigning of the machine 1 to a new patient; assigning of the machine 1 to a new organization, such as a physician or operator or homecare provider; expiration of a defined period of time in which no data accessing has occurred; access by a new or additional user to the machine 1 or the data received from it; critical changes to the machine 1, such as replacement of components, changing of therapy settings, update of program code in the machine 1; use of a new or additional data interface or output device or display device or a type of data transmission between machine 1 and remote station 3; critical changes to the remote station 3, such as changing of program code, changing of location, changing of electronic components.

In another embodiment, for certain actions basically a new authorization code is required each time in the form of a onetime code. Such actions or parameters are, for example: assignment of a patient to a machine 1; changing of configuration parameters of a machine 1; updating of the program code of a machine 1; reading out of data volumes exceeding the normally transmitted level and therefore causing elevated transmission costs; transmission of identifying therapy data; assignment of a patient to an organization, such as an attending physician or provider; erasing of data or patient records.

In one possible embodiment, the respirator machine 1 is only able to generate or receive or store or output authorization codes by updating of the program code.

This affords substantial advantages, especially with respect to permanent coding possibilities in the form of a label affixed to the outside of the machine 1. Thus, it is made possible to create and use the machine 1 at first without the function of administering of authorization codes. Only afterwards is this functionality brought into the machine 1 if so required.

In an especially advantageous embodiment, in addition with the aid of a code stored in particular permanently in the machine 1 (e.g., a certificate) and a code stored in particular permanently in the remote station 3 (e.g., another certificate) a check is first made to make sure that respirator machine 1 and remote station 3 are in fact authentic products and not simulations meant to circumvent or eavesdrop on the authorization code. Before a data communication occurs between respirator machine 1 and remote station 3, the latter mutually verify their certificates or the two of them communicate with an additional check point, such as a certificate server, which confirms that the certificates are genuine.

In an especially advantageous embodiment, the user action involves at least one registration of the user vis-à-vis the remote station 3. Here, the authorization code may only be requested by a registered user.

For the registration, the user at first sets up a secure account on a provided server 13, for example, where he authenticates himself. Once the user has logged in to the server 13, he may create a storage medium 41 or data medium (such as an SD card) with data which uniquely identify him as the user. For this, the server 13 saves a user certificate on the storage medium 41, which thereby becomes a “key card”. In particular, the certificate consists of at least one code which identifies the user and which is secured, e.g., by a check sum.

Optionally, the certificate or the code may contain further information. For example, an attribute of the user group: is the user a physician, a patient, or a provider?

The certificate may also have at least one onetime code, so that a key card if lost for example can be declared invalid on the server 13 and a new, distinguishable key card can be created.

The certificate may contain at least one attribute as to whether the key card should be valid only for one machine 1 or permanently valid.

The certificate may comprise at least one server address or a dial-in address to the internet, which exactly leads to the database on which the respective holder of the key card has his account.

The user inserts the key card with the patient present into the respirator machine 1 upon placing it in operation or at the time of remote initialization. The respirator machine 1 recognizes the user certificate on the key card and sends a message to the remote station 3 or the server. This message contains at least one portion of the user certificate identifying the user and a code identifying the machine, e.g., a serial number and/or a machine identification number.

The server then checks the validity of the two identities (user certificate and machine identification). If both are valid, the machine 1 and the user are brought together, without any code needing to be entered manually. The user will find the machine 1 the next time he logs in to the remote station 3 or the server and can view the therapy data of the machine 1 or assign a patient to the machine 1.

It may be provided that other users or other remote stations 3 may access the machine 1. Either automatically, e.g., a user group to which the user who inserted the key card into the machine 1 belongs, or other users, e.g., physicians, whom the original user selects manually for the patient.

The machine 1 decides, preferably with the aid of the attribute of the user certificate, whether to then erase or invalidate it or keep it unchanged on the key card for other machines.

The user may also be the patient who wishes to view his own therapy data via the remote station 3 or on the server 13. He may gain access to the data of his machine 1 via a key card in exactly the same way.

The data processing device 2 or the server 13 of the remote station 3 preferably decides whether it requires a new certificate, triggered by a particular event, especially from the patient. In particular, events are taken into consideration which indicate a change of patient. Such events may be recognized, e.g., by means of characteristic parameters. Such parameters are, e.g., therapy data erased in the machine 1, a lengthy time of no use of the machine 1, an assignment of the machine 1 on the server to a new patient.

The invention proposed here enables an especially secure access to therapy data and offers reliable protection against mix-ups or reading out or writing data into the wrong machine 1. Furthermore, it offers protection against persons deliberately gaining access to the reading out or writing in of data for respirator machines 1 to which they have no authorization. It also effectively prevents any person from accessing all the therapy data of a respirator machine 1 even if they only possess an authorization or the required knowledge for a portion of the data.

### LIST OF REFERENCE NUMBERS

1 Respirator machine
2 Data processing device
3 Remote station
10 Respiration system
11 Display device
12 Code generator
13 Server
21 Monitoring device
23 Personal computer
31 Storage device
41 Storage medium
51 Transmission device
61 User interface
100 Respirator device
101 Blower device
102 Breathing interface
103 Operating elements
105 Breathing mask
106 Headgear
107 Connection element
108 Exhalation element
109 Connection hose
110 Pressure measuring hose
111 Inlet nozzle
112 Connection device

What is claimed is:

1. A method for operating at least one data processing device of a respiration system comprising at least one respirator machine and at least one remote station physically separated from the respirator machine,
wherein therapy data are at least partly transmissible by the data processing device between the respirator machine and the remote station and wherein at least one authorization code is stored by at least one user of the respiration system in the data processing device to obtain authorized access to the therapy data via the remote station,
and wherein
the authorization code is only provided to the at least one user when the latter requests the authorization code by at least one user action to be performed vis-à-vis the data processing device.

2. The method of claim 1, wherein the user action is performed at least in part with the respirator machine and wherein the authorization code only authorizes an access to the therapy data of that respirator machine with which the user action is performed.

3. The method of claim 1, wherein the user action comprises at least one placement of the respirator machine in operation.

4. The method of claim 1, wherein the user action comprises at least one biometrical detection of the user and/or at least one voice command and/or at least one connecting of the respirator machine to at least one storage medium.

5. The method of claim 1, wherein the requested authorization code is indicated to the user on at least one display device and/or stored on at least one storage medium.

6. The method of claim 1, wherein the user action comprises at least one registration of the user vis-à-vis the data processing device so that only a registered user can request the authorization code.

7. The method of claim 1, wherein the user upon registration is assigned at least one certificate and wherein the certificate must be presented to the respirator machine in order to be able to request the authorization code.

8. The method of claim 7, wherein the certificate is stored on a portable storage medium and wherein the authorization code may only be requested when the respirator machine is connected to the storage medium.

9. The method of claim 8, wherein the remote station in addition to the certificate also stores at least one device certificate identifying the respirator machine on the storage medium.

10. The method of claim 7, wherein the authorization code is generated at least partly by the respirator machine with help of the certificate.

11. The method of claim 6, wherein access to therapy data is released when a registered user logs in to the remote station after having first requested the authorization code as a registered user.

12. The method of claim 1, wherein access to therapy data is only released when the at least one user has communicated the authorization code provided to her to the remote station.

13. The method of claim 1, wherein the authorization code is entered by the at least one user in the remote station.

14. The method of claim 1, wherein the respirator machine relays the authorization code at least partly independently to the remote station.

15. The method of claim 1, wherein the authorization code comprises at least one serial number and/or at least one device identification number of the respirator machine or is generated at least in part from the latter.

16. The method of claim 1, wherein the authorization code comprises at least one piece of information about an authorization of the user with respect to access to therapy data and/or at least one piece of information about at least one read right and/or a write right of the user.

17. The method of claim 1, wherein the authorization code has at least one time-limited validity and/or a onetime validity

18. The method of claim 1, wherein a validity of the authorization code is connected to the user.

19. The method of claim 1, wherein the authorization code which is requested by the at least one user is at least partly stored in the respirator machine and/or is at least partly generated by the respirator machine.

20. A respiration system, wherein the system comprises at least one respirator machine comprising at least one respirator device to create an air flow for a respiratory therapy, at least one remote station physically separated from the respirator machine, and at least one data processing device
which is capable of sending therapy data between the respirator machine and the remote station,
wherein at least one authorization code can be stored in the data processing device in order to grant a user authorized access to the therapy data via the remote station,
the data processing device being suitable and designed for providing the user the authorization code when the latter requests the authorization code by at least one user action to be performed vis-à-vis the data processing device.
